(19) United States
(12) Patent Application Publication
(10) Pub. No.: US 2025/0041298 A1
(43) Pub. Date: Feb. 6, 2025

(54) CDK4 INHIBITOR FOR THE TREATMENT OF CANCER

(71) Applicants: Pfizer Inc., New York, NY (US); PFIZER INC., New York, NY (US)

(72) Inventors: Tun Tun Lin, San Diego, CA (US); Jing Yang, Bellevue, WA (US)

(73) Assignees: Pfizer Inc., New York, NY (US); PFIZER INC., New York, NY (US)

(21) Appl. No.: 18/714,642

(22) PCT Filed: Nov. 29, 2022
(86) PCT No.: PCT/IB2022/061525

§ 371 Date: May 30, 2024

Related U.S. Application Data
(60) Provisional application No. 63/285,320, filed on Dec. 2, 2021.
(60) Provisional application No. 63/382,346, filed on Nov. 4, 2022.
(60) Provisional application No. 63/383,969, filed on Nov. 16, 2022.

(51) Int. Cl. A61K 31/506; A61K 31/4196; A61K 31/565; A61P 35/00

(52) U.S. Cl. CPC A61K 31/506; A61K 31/4196; A61K 31/565; A61P 35/00

(57) ABSTRACT

The disclosure provides a method of treating cancer comprising administering to a subject in need thereof with a therapeutically effective amount of PF-07220060. The disclosure also provides a method of treating cancer comprising administering to a subject in need thereof with a therapeutically effective amount of PF-07220060 and an endocrine therapy agent.

### FIELD OF THE INVENTION

The present disclosure relates to the therapeutic treatment of cancer with a cyclin-dependent kinase (CDK) inhibitor, 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (hereinafter PF-07220060) or a pharmaceutically acceptable salt thereof, either alone as a monotherapy or in combination with endocrine therapeutics.

The invention also relates to associated combination therapies, pharmaceutical compositions, and pharmaceutical uses.

### INTRODUCTION

CDKs are important cellular enzymes that perform essential functions in regulating eukaryotic cell division and proliferation. CDK inhibitors may be useful for the treatment of proliferative disorders, including cancer.

Cancer develops through unregulated cell proliferation, which is reflected by unchecked progression of the cell cycle, a loss of checkpoint controls subsequently permitting uncontrolled cellular proliferation. Rb1 (retinoblastoma protein) is a crucial negative regulator through G1 and quiescent G0 phases of the cell cycle until and unless phosphorylated by CDK4 or CDK6. (Weinberg, R. A. The retinoblastoma protein and cell cycle control. Cell, vol. 81, 323-330, 1995). The notion of dysregulation of the CDK4/6-retinoblastoma protein axis in cancer is supported by gene alterations commonly identified in breast cancers, such as cyclin D1 (CCND1) and cyclin E1 (CCNE1) gene amplification and alterations in endogenous CDK4-cyclin D1 inhibitor p16 (INK4a, encoded by the CDKN2A gene), TP53 and PIK3CA genes. (Cancer Genome Atlas Network. Comprehensive genomic characterization of human breast tumours. Nature, 490, 61-70, 2012).

CDK4/6 inhibition has emerged as a promising strategy for cancer therapy, especially for the treatment of endocrine resistant BC. (Rani, A., et. al., Endocrine Resistance in Hormone Receptor Positive Breast Cancer-From Mechanism to Therapy. Front Endocrinol (Lausanne) 10:245, 2019). CDK4/6 inhibitors (e.g., palbociclib, abemaciclib, ribociclib) when dosed in combination with endocrine therapy, have significantly improved progression-free survival and/or overall survival for patients with HR-positive/HER2-negative metastatic breast cancer. (Spring, L. M., et. al., Cyclin-dependent kinase 4 and 6 inhibitors for hormone receptor-positive breast cancer: past, present, and future. Lancet, 395, 817-827, 2020).

Although CDK4/6 inhibitors increase response rates and prolong disease control in patients with HR+, HER2-breast cancer, they are associated with dose-limiting hematologic toxicities, primarily neutropenia.

PF-07220060 is a potent inhibitor of cyclin dependent kinase 4 (CDK4). Preparation of PF-07220060 is described in International Patent Publication No. WO 2019/207463 and U.S. Pat. No. 10,766,884. The contents of each of the foregoing documents are incorporated herein by reference in their entirety. PF-07220060 differs from currently approved dual CDK4/6 inhibitors in that it displays greater CDK4-over-CDK6 selectivity. In in vivo models, PF-07220060 reduces dose-limiting neutropenia and is projected to potentially attain higher tolerated plasma concentrations than dual CDK4/6 inhibitors, thus potentially enabling increased inhibition of the CDK4 oncogene in tumors.

There is a need for appropriate and efficacious dosing regimens of PF-07220060 as a single agent and in combination therapies for treating cancers while minimizing adverse events.

### SUMMARY

The disclosure relates to both single agent and combination therapies, for treating cancer, which comprise the CDK4 inhibitor, PF-07220060, or a pharmaceutically acceptable salt thereof.

In certain embodiments, the disclosure provides a method of treating cancer comprising orally administering to a subject in need thereof a therapeutically effective amount of PF-07220060, or a pharmaceutically acceptable salt thereof. Particularly, the method includes administering to the subject a pharmaceutical composition comprising a therapeutically effective amount of PF-07220060 in a total daily dose of from about 200 mg to about 1000 mg per day, in certain embodiments, from about 100 mg to about 500 mg twice per day (BID).

In certain embodiments, the disclosure provides a method of treating cancer comprising administering to a subject in need thereof with a therapeutically effective amount of PF-07220060, or a pharmaceutically acceptable salt thereof, and an endocrine therapy agent. In embodiments, the endocrine therapy agent is an aromatase inhibitor, a selective estrogen receptor degrader (SERD), or a selective estrogen receptor modulator (SERM). In embodiments, the endocrine therapy agent includes fulvestrant, tamoxifen, toremifene, anastrozole, exemestane, or letrozole.

Accordingly, embodiments herein provide dosing regimens for PF-07220060 as a single agent and in combination therapies for treating cancer, by which adverse effects are minimized in a subject during the treatment period.

### BRIEF DESCRIPTION OF THE FIGURES

FIG. 1 shows the PF-07220060 plasma concentration vs. nominal time profiles on Cycle 1 Day 1 following oral dose administration of PF-07220060 as monotherapy, or in combination with letrozole or fulvestrant.

FIG. 2 shows the PF-07220060 plasma concentration vs. nominal time profiles on Cycle 1 Day 15 following repeated BID oral dose administration of PF-07220060 as monotherapy, or in combination with letrozole or fulvestrant.

### DETAILED DESCRIPTION

The present disclosure may be understood more readily by reference to the following detailed description of the aspects and embodiments of the disclosure and the Examples included herein. It is to be understood that the terminology used herein is for describing specific embodiments only and is not intended to be limiting. It is further to be understood that unless specifically defined herein, the terminology used herein is to be given its traditional meaning as known in the relevant art.

### Definitions

As used herein, the singular form “a”, “an”, and “the” include plural references unless indicated otherwise. For example, “a” substituent includes one or more substituents.

The term “about” means having a value falling within an accepted standard of error of the mean, when considered by one of ordinary skill in the art. In some embodiments, the term “about” means within +10% of the indicated value. For example, a dose of about 400 mg should be understood to mean that the dose may vary between 360 mg and 440 mg.

The disclosure described herein may be suitably practiced in the absence of any element(s) not specifically disclosed herein. Thus, for example, in each instance herein any of the terms “comprising”, “consisting essentially of”, and “consisting of” may be replaced with either of the other two terms.

As used herein, “dose limiting toxicity” (DLT) refers to the dosage of PF-07220060 that is contraindicative of a further increase in dosage.

As used herein, “measurable lesion” refers to Lesions that can be accurately measured in at least one dimension, lesions with longest diameter twice the slice thickness and at least 10 mm or greater when assessed by CT or MRI (slice thickness 5-8 mm), lesions with longest diameter at least 20 mm when assessed by Chest X-ray, superficial lesions with longest diameter 10 mm or greater when assessed by caliper, or malignant lymph nodes with the short axis 15 mm or greater when assessed by CT.

As used herein “maximum tolerated dose” (MTD) refers to the highest dosage of PF-07220060 that does not cause unacceptable side effects or intolerable toxicities. MTD is estimated using the mTPI based on observed DLT rate, with a target DLT rate of 27.5% and equivalence interval of 22.5-32.5%.

As used herein, the term “pharmaceutically acceptable carrier” or “pharmaceutically acceptable excipient” refers to a component that may be included in the compositions described herein, is physiologically suitable for pharmaceutical use, and causes no significant adverse effects to a subject.

As used herein, a “rest period” is the number of days from administration of one complete dose of the active agent to the next administration of one complete dose of the active agent.

As used herein, the term “week” means 7 consecutive days. Thus, a 4-week period is 28 consecutive days starting on any day of the calendar week.

As used herein, the term “subject” may be a mammal, which refers to any animal species of the Mammalia class. Examples of mammals include: humans; non-human primates such as monkeys; laboratory animals such as rats, mice, guinea pigs; domestic animals such as cats, dogs, rabbits, cattle, sheep, goats, horses, and pigs; and captive wild animals such as lions, tigers, and the like. In some embodiments, the subject is a human. In some embodiments, the subject is a female. In some embodiments, the subject is a male.

As used herein, the phrase “therapeutically effective amount” for use and/or for treating a subject refers to an amount that provides, in single or multiple doses, alone, or in combination with one or more other agents, treatments, protocols, or therapeutic regimens, a detectable response of any duration of time (transient, medium or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an active agent effective for producing a desired therapeutic effect upon administration to a subject, for example, to stem the growth, or result in the shrinkage, of a cancerous tumor.

Determination of a therapeutically effective amount is well within the capability of those skilled in the art. In various embodiments, the dosage can vary within the range depending upon the dosage form employed and the route of administration utilized. In some embodiments, a therapeutically effective amount of a compound described herein administered to a subject can be dependent upon factors known to a skilled artisan, including bioactivity and bioavailability of the compound (e.g., half-life and stability of the compound in the body), chemical properties of the compound (e.g., molecular weight, hydrophobility and solubility); route and frequency of administration, and the like. Further, it will be understood that the specific dose of the pharmaceutical composition comprising a compound as disclosed herein can depend on a variety of factors including physical condition of the subject (e.g., age, gender, weight), and medical history of the subject (e.g., medications being taken, health condition other diseases or disorders). The precise dose of a pharmaceutical composition administered to a subject can be determined by methods known to a skilled artisan such as a pharmacologist, or an anesthesiologist.

As used herein, the term “ameliorate” refers to any reduction in the extent, severity, frequency, and/or likelihood of a symptom or clinical sign characteristic of a particular disease. “Symptom” refers to any subjective evidence of disease or of a subject's condition.

As used herein, “treat” or “treating” a cancer and/or a cancer-associated disease means to administer a mono- or combination therapy according to the present disclosure to a subject, patient or individual having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50: 1S-10S (2009)).

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukaemia's (cancers of the blood) generally do not form solid tumors (National Cancer Institute, Dictionary of Cancer Terms).

As used herein, the terms “combination” or “combination therapy” refer to the administration of two or more therapeutic agents of the combination therapy, either alone or in the form of a pharmaceutical composition or medicament. The combination therapy may be administered sequentially, concurrently or simultaneously.

When administering a combination therapy of two or more agents, the agents may be administered on the same treatment cycle or using different cycles. In a preferred embodiment, PF-07220060 is administered continuously on a 28-day cycle. Similarly, letrozole is typically administered continuously on a 28-day treatment cycle. Palbociclib is typically administered using an intermittent 28-day treatment cycle, comprising administration of the drug for 21 days, with a rest period of 7 days between the cycles. Fulvestrant is typically administered intramuscularly on days 1, 15, 29 of the first treatment cycle and once monthly thereafter.

Each therapeutic agent of the methods and combination therapies described herein may be administered either alone, or in a medicament (also referred to herein as a pharmaceutical composition) which comprises the therapeutic agent and one or more pharmaceutically acceptable carriers, excipients, or diluents, according to pharmaceutical practice.

The term “sequential” or “sequentially” refers to the administration of each therapeutic agent of the combination therapy, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

The term “concurrently” refers to the administration of each therapeutic agent in a combination therapy, either alone or in separate medicaments, wherein the second therapeutic agent is administered immediately after the first therapeutic agent, but that the therapeutic agents can be administered in any order. In a preferred embodiment the therapeutic agents are administered concurrently.

The term “simultaneous” refers to the administration of each therapeutic agent of the combination therapy in the same medicament, for example as a fixed dose combination comprising two or more drugs in a single dosage form.

A “dosing regimen” refers to the period of administration of one or more drugs, compounds or compositions, comprising one or more treatment cycles, wherein each treatment cycle may include administration of one or more agents at different times, frequencies or amounts, using the same or different routes of administration. Repetition of the administration or dosing regimens, or adjustment of the administration or dosing regimen may be conducted as necessary to achieve the desired treatment effect.

PF-07220060 (Pfizer Inc.) is a selective CDK4 inhibitor that is currently in phase I/Ib clinical trial for the treatment of cancers, and has the following structure of formula (I):

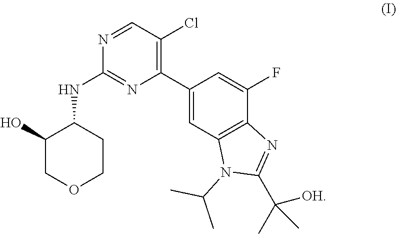 [SMILES: CC(C)n1c(C(C)(C)O)nc2c(F)cc(-c3nc(N[C@@H]4CCOC[C@H]4O)ncc3Cl)cc21]

Accordingly, certain embodiments of this disclosure provide a therapeutic dose and dosing regimen comprising administering to a subject a therapeutically effective amount of PF-07220060.

PF-07220060 may be present in a pharmaceutical composition which includes a pharmaceutically acceptable carrier. The therapeutically effective amount of PF-07220060 in the pharmaceutical compositions can be from about 200 mg to about 1000 mg, or any of the therapeutically effective amounts disclosed herein.

In certain embodiments, the therapeutically effective amount of PF-07220060 is from about 200 mg to about 1000 mg per day (i.e., total daily dose), for example, from about 200 mg to about 500 mg, from about 200 mg to about 450 mg, from about 200 mg to about 400 mg, from about 200 mg to about 350 mg, from about 200 mg to about 300 mg, from about 400 mg to about 950 mg, from about 400 mg to about 900 mg, from about 400 mg to about 850 mg, from about 400 mg to about 800 mg, from about 500 mg to about 950 mg, from about 500 mg to about 900 mg, from about 500 mg to about 850 mg, from about 500 mg to about 800 mg, from about 600 mg to about 950 mg, from about 600 mg to about 900 mg, from about 600 mg to about 850 mg, or from about 600 mg to about 800 mg per day.

In certain embodiments, PF-07220060 is administered in doses of from about 200 mg to about 1000 mg once a day (QD), for example, from about 200 mg to about 500 mg QD, from about 200 mg to about 450 mg QD, from about 200 mg to about 400 mg QD, from about 200 mg to about 350 mg QD, from about 200 mg to about 300 mg QD, from about 400 mg to about 950 mg QD, from about 400 mg to about 900 mg QD, from about 400 mg to about 850 mg QD, from about 400 mg to about 800 mg QD, from about 500 mg to about 950 mg QD, from about 500 mg to about 900 mg QD, from about 500 mg to about 850 mg QD, from about 500 mg to about 800 mg QD, from about 600 mg to about 950 mg QD, from about 600 mg to about 900 mg QD, from about 600 mg to about 850 mg QD, or from about 600 mg to about 800 mg QD.

In certain embodiments, the therapeutically effective amount of PF-07220060 is from about 100 mg to about 500 mg twice a day (BID), for example, from about 200 mg to about 500 mg BID, from about 250 mg to about 500 mg BID, from about 300 mg to about 500 mg BID, from about 350 mg to about 500 mg BID, from about 400 mg to about 500 mg BID, from about 100 mg to about 450 mg BID, from about 150 mg to about 450 mg BID, from about 200 mg to about 450 mg BID, from about 250 mg to about 450 mg BID, from about 300 mg to about 450 mg BID, from about 350 mg to about 450 mg BID, from about 400 mg to about 450 mg BID, from about 100 mg to about 400 mg BID, from about 150 mg to about 400 mg BID, from about 200 mg to about 400 mg BID, from about 250 mg to about 400 mg BID, from about 300 mg to about 400 mg BID, from about 350 mg to about 400 mg BID.

In some embodiments, the subject is administered PF-07220060 at a dose of any of the therapeutically effective amounts disclosed herein.

The pharmaceutical composition comprising the therapeutically effective amount of PF-07220060 described herein may be administered once a day (QD) or twice a day (BID).

In some embodiments, the subject is administered PF-07220060 at a dose of from about 200 mg to about 1000 mg per day, for example, in daily doses of about 200 mg, about 300 mg, about 400 mg, about 500 mg, about 600 mg, about 700 mg, about 800 mg, about 900 mg, or 1000 mg.

In some embodiments, PF-07220060 is administered in doses of about 200 mg QD, about 300 mg QD, about 400 mg QD, about 500 mg QD, about 600 mg QD, about 700 mg QD, about 800 mg QD, about 900 mg QD, or 1000 mg QD.

In some embodiments, PF-07220060 is administered in doses of about 100 mg BID, about 200 mg BID, about 300 mg BID, about 400 mg BID, or about 500 mg BID.

The amount of PF-07220060 administered may be increased or decreased based on the weight, age, health, sex, or medical condition of the subject. One of skill in the art would be able to determine the proper dose for a subject based on this disclosure.

PF-07220060 may be administered in treatment cycles, with or without rest periods in between the treatment cycles. A treatment cycle may be a duration of about 7 days, about 14 days, about 21 days, about 28 days, about 35 days and so on, or any days in between. A rest period can be one day, a few days (e.g., 1 day, 2 days, 3 days, 4 days, 5 days, 6 days, and so on), one week, several weeks (e.g., 2 weeks, 3 weeks and so on), or any days in between (e.g., 1 week and 3 days). In some embodiments, PF-07220060 is administered continuously without any rest period in between (i.e., continuous treatment until termination). In some embodiments, PF-07220060 is administered for a treatment cycle (e.g., about 28 days) with or without a rest period. In some embodiments, PF-07220060 is administered for about 28 days with a rest period of about one week. PF-07220060 may be administered for at least about 7 days, about 14 days, about 21 days, about 28 days, about 2 months, about 3 months, about 12 months, about 24 months, and more.

The pharmaceutical compositions may be administered with or without food.

The pharmaceutical compositions may be administered by one or more routes as considered appropriate by a skilled person in the art and depending on the dosage form. Formulation of drugs is discussed in Remington's Pharmaceutical Sciences, 18th Ed., (1995) Mack Publishing Co., Easton, Pa. Other examples of drug formulations can be found in Liberman, H. A. and Lachman, L., Eds., Pharmaceutical Dosage Forms, Marcel Decker, Vol 3, 2nd Ed., New York, N.Y. Where the compound is administered orally, it may be formulated as a pill, capsule, tablet, etc. with a pharmaceutically acceptable carrier, glidant, or excipient.

The pharmaceutical compositions may be in one or more dosage forms (e.g., capsule, liquid, tablet, powder).

In some embodiments, the pharmaceutical compositions may be administered in an immediate release formulation or a modified release formulation.

By “immediate release” or “IR” is meant broadly an oral dosage form formulated to release an API immediately after oral administration. In IR formulations, no deliberate effort is made to modify the drug release rate.

### Therapeutic Methods and Uses

In certain embodiments, the disclosure provides a method for treating cancer of a subject in need thereof, which includes administering to the subject a therapeutically effective amount of PF-07220060 as described herein.

In certain embodiments, the disclosure also provides a method for treating cancer of a subject which includes administering to the subject a therapeutically effective amount of PF-07220060 as described herein and an endocrine therapy agent.

In certain embodiments, the disclosure provides a use of PF-07220060 in the manufacture of a medicament for use in the treatment of cancer in a subject in need thereof wherein the medicament is administered in a therapeutically effective unit dosage of PF-07220060 as described herein.

In certain embodiments, the disclosure provides a use of PF-07220060 together with endocrine therapy in the manufacture of a medicament for use in the treatment of cancer in a subject in need thereof wherein the medicament is administered in a therapeutically effective unit dosage of PF-07220060 as described herein.

In certain embodiments, the disclosure provides a medicament comprising a therapeutically effective amount of PF-07220060 as described herein for use in treating cancer in a subject in need thereof.

In embodiments, the disclosure provides a medicament comprising a therapeutically effective amount of PF-07220060 as described herein and an endocrine therapy agent for use in treating cancer in a subject in need thereof.

In certain embodiments of each of the methods, medicaments, combinations and uses described herein, PF-07220060 is administered continuously (i.e., daily).

In certain embodiments, the method disclosed herein includes administering PF-07220060 to a subject who is suffering from cancer that is mediated by CDK4. In some embodiments, the cancer is characterized by amplification or overexpression of CDK4 and/or CCND1. In one embodiment, the cancer is characterized by amplification or overexpression of CDK4. In one embodiment, the cancer is characterized by amplification of the CCND1 gene or overexpression of cyclin D1.

In certain embodiments, the method disclosed herein includes administering PF-07220060 to a subject who is suffering from cancer that is selected from breast cancer, ovarian cancer, fallopian tube cancer, primary peritoneal cancer (PPC), bladder cancer, uterine cancer, prostate cancer, lung cancer (including non-small-cell lung carcinoma (NSCLC)), small-cell lung carcinoma (SCLC), squamous cell carcinoma or adenocarcinoma), esophageal cancer, head and neck cancer (including head and neck squamous cell carcinoma (HNSCC), colorectal cancer (CRC), kidney cancer (including renal cell carcinoma (RCC)), liver cancer (including hepatocellular carcinoma (HCC)), pancreatic cancer, stomach (i.e., gastric) cancer, endometrial cancer, liposarcoma, and thyroid cancer. In further embodiments of the methods provided herein, the cancer is selected from the group consisting of breast cancer, ovarian cancer, bladder cancer, uterine cancer, prostate cancer, lung cancer, esophageal cancer, liver cancer, pancreatic cancer, stomach cancer, or combinations thereof.

In some embodiments, the cancer is advanced or metastatic solid tumors.

In some embodiments, the cancer is NSCLC. In some embodiments, the cancer is adenocarcinoma of NSCLC. In some embodiments, the cancer is prostate cancer. In some embodiments, the cancer is colorectal cancer. In some embodiments, the cancer is liposarcoma.

In some embodiments, the cancer is breast cancer. In some embodiments, the breast cancer is advanced or metastatic breast cancer. In some embodiments, the breast cancer is locally advanced. In some embodiments, the breast cancer is metastatic breast cancer. In some embodiments, the breast cancer is hormone receptor positive (HR+), i.e., the breast cancer is estrogen receptor positive (ER+) and/or progesterone receptor positive (PR+). In some embodiments, the breast cancer is hormone receptor negative (HR−), i.e., the breast cancer is estrogen receptor negative (ER−) and progesterone receptor negative (PR−). In some embodiments, the breast cancer is human epidermal growth factor receptor 2 negative (HER2−). In some embodiments, the breast cancer is human epidermal growth factor receptor 2 positive (HER2+). In some embodiments, the breast cancer is HR+/HER2− breast cancer. In some embodiments, the breast cancer is HR−/HER2+ breast cancer. In some embodiments, the breast cancer is ER+/HR+. In some embodiments, the breast cancer is ER+/HER2−. In some embodiments, the breast cancer is triple negative breast cancer (TNBC), i.e., the breast cancer is ER−, PR− and HER2−.

In some embodiments, the breast cancer is endocrine resistant breast cancer, trastuzumab or pertuzumab resistant breast cancer, or breast cancer demonstrating primary or acquired resistance to CDK4/CDK6 inhibition. In some embodiments, the breast cancer is resistant to treatment with a standard of care agent; for example, the breast cancer may demonstrate primary or acquired resistance to endocrine therapy, HER2-targeted agents (e.g., tamoxifen, trastuzumab emtansine, fam-trastuzumab deruxtecan, pertuzumab, lapatinib, neratinib or tucatinib), or CDK4/6 inhibitors. In some embodiments, the subject is refractory to endocrine therapy.

In some embodiments, the breast cancer is refractory or resistant to treatment with, or has progressed on, treatment with antineoplastic chemotherapeutic agents such as platinum agents, taxanes, anthracyclines or anti-metabolites.

In some embodiments, the breast cancer has progressed during treatment or within 12 months of completion of adjuvant therapy with an aromatase inhibitor. In some embodiments, the breast cancer has progressed during treatment or within 12 months of completion of adjuvant therapy with tamxifen.

In some embodiments, PF-07220060 is administered as first line therapy. In other embodiments, PF-07220060 is administered as second (or later) line therapy. In some embodiments, PF-07220060 is administered as second (or later) line therapy following treatment with an endocrine therapeutic agent and/or a CDK4/CDK6 inhibitor. In some embodiments, PF-07220060 is administered as second (or later) line therapy following treatment with an endocrine therapeutic agent, such as, an aromatase inhibitor; a selective estrogen receptor modulator (SERM), e.g., tamoxifen; or a selective estrogen degrader/downregulator (SERD). In some embodiments, PF-07220060 is administered as second (or later) line therapy following treatment with one or more chemotherapy regimens. In some embodiments, PF-07220060 is administered as second (or later) line therapy following treatment with HER2 targeted agents.

In certain embodiments, the method disclosed herein further includes administering to a subject a therapeutically effective amount of PF-07220060 and an endocrine therapy agent. An “endocrine therapy agent” is a biological (large molecule) or chemical (small molecule) compound useful in the treatment of cancer, regardless of mechanism of action.

In some embodiments, the endocrine therapy agent is an aromatase inhibitor, an androgen receptor inhibitor, a selective estrogen receptor degrader (SERD), or a selective estrogen receptor modulator (SERM). In some embodiments, the endocrine therapy agent is an androgen receptor inhibitor. In some embodiments, the endocrine therapy agent is an aromatase inhibitor. In some such embodiments, the aromatase inhibitor is selected from the group consisting of letrozole, anastrozole and exemestane. In one embodiment, the aromatase inhibitor is letrozole. In some embodiments, the endocrine therapy agent is a SERD. In some such embodiments, the SERD is selected from the group consisting of fulvestrant, elacestrant (RAD-1901, Radius Health/Menarini), amcenestrant (SAR439859, Sanofi), giredestrant (GDC9545, Roche), RG6171 (Roche), camizestrant (AZD9833, AstraZeneca), AZD9496 (AstraZeneca), rintodestrant (G1 Therapeutics), ZN-c5 (Zentalis), LSZ102 (Novartis), D-0502 (Inventisbio), LY3484356 (Eli Lilly), and SHR9549 (Jiansu Hengrui Medicine). In some embodiments, the SERD is fulvestrant. In some embodiments, the endocrine therapy agent is a SERM. In some such embodiments, the SERM is selected from the group consisting of tamoxifen, raloxifene, toremifene, lasofoxifene, bazedoxifene and afimoxifene. In some such embodiments, the SERM is tamoxifen or raloxifene. In preferred embodiments, the endocrine therapy agent is letrozole or fulvestrant.

The endocrine therapy agent may be administered according to the standard of care per package insert or provided by the health care professionals. The term “package insert” refers to instructions customarily included in commercial packages of therapeutic products, that contain information about the indications, usage, dosage, administration, contraindications and/or warnings concerning the use of such therapeutic products.

In certain embodiments, the endocrine therapy agent is administered to the subject during the course of the treatment with PF-07220060. In certain embodiments, the first dose of the endocrine therapy agent is administered prior to administering the first dose of PF-07220060. In certain embodiments, the first dose of the endocrine therapy agent is administered on the same day as administering the first dose of PF-07220060. In certain embodiments, the first dose of the endocrine therapy agent is administered after the start of the treatment with PF-07220060.

In certain embodiments, prior to the administration of PF-07220060 to the subject, the subject has been previously treated with one or more lines of endocrine therapy.

In certain embodiments, prior to the administration of PF-07220060 to the subject, the subject has been previously treated with chemotherapy, radiotherapy, and/or surgical resection.

In certain embodiments, prior to the administration of PF-07220060 to the subject, the subject has been previously treated with a CDK4/6 inhibitor.

In some embodiments, the present treatment results in complete response (CR), partial response (PR), or stable disease (SD) in the subject.

### Treatment Outcomes and Tumor Response Evaluation

The present therapy may result in a complete response (CR), partial response (PR), progressive disease (PD), or stable disease (SD) in patients. Bioimaging can be used to evaluate the level of response to the therapy. Cytology and histology may also be used as needed (e.g., to locate any residual lesions). In some embodiments, the various levels of response can be evaluated in accordance with Table 1 below.

TABLE 1
Evaluation of Target Disease

| Response | |
|---|---|
| Classification | Criteria |
| Complete | Complete disappearance of all target lesions with the |
| Response | exception of nodal disease. All target nodes must have |
| (CR) | reduction in short axis to <10 mm). |
| Partial | At least a 30% decrease in the sum of diameters of all |
| Response | target measurable lesions, taking as reference the |
| (PR) | baseline of the sum of diameters. The short diameter is |
| | used in the sum for target nodes, while the longest |
| | diameter is used in the sum for all other target lesions. |
| Progressive | 20% increase in the sum of diameters of target |
| Disease (PD) | measurable lesions above the smallest sum observed |
| | (over baseline if no decrease in the sum is observed |
| | during therapy), with a minimum absolute increase of |
| | 5 mm. |
| Stable | Neither sufficient shrinkage to qualify for PR nor |
| Disease (SD) | sufficient increase to qualify for PD, taking as reference |
| | the smallest sum diameters while on study. Stable can |
| | follow PR only in the rare case that the sum increases |
| | by less than 20% from the nadir, but enough that a |
| | previously documented 30% decrease no longer holds. |
| Non-CR/ | Persistence of any non-target lesions and/or tumor |
| Non-PD | marker level bove the normal limits (<10 mm short |
| | axis) |
| Not- | Progression has not been documented and one or more |
| evaluable | of the followings occurs: (1) one or more target |
| (NE) | measurable lesions have not been assessed; (2) one or |
| | more target lesions cannot be measured accurately |
| | (e.g., poorly visible unless due to being too small to |
| | measure); or one or more target lesions were excised or |
| | irradiated. |

The present disclosure provides a number of exemplary embodiments. Non-limiting exemplary embodiments of the present disclosure are shown below.

Embodiment 1. A method of treating cancer comprising orally administering to a subject in need thereof a therapeutically effective amount of PF-07220060, or a pharmaceutically acceptable salt thereof, wherein the therapeutically effective amount is from about 100 mg to about 500 mg twice per day (BID).

Embodiment 2. The method of embodiment 1, wherein the therapeutically effective amount is from about 300 mg to about 500 mg BID.

Embodiment 3. The method of embodiment 1 or 2, wherein the therapeutically effective amount is about 300 mg BID.

Embodiment 4. The method of embodiment 1 or 2, wherein the therapeutically effective amount is about 400 mg BID.

Embodiment 5. The method of any one of embodiments 1 to 4, wherein PF-07220060 is administered continuously.

Embodiment 6. The method of any one of embodiments 1 to 5, wherein PF-07220060 is administered in a tablet or capsule form.

Embodiment 7. A method of treating cancer comprising administering to a subject in need thereof a therapeutically effective amount of a pharmaceutical composition comprising PF-07220060 and an endocrine therapy agent to a subject in need thereof, wherein the therapeutically effective amount of PF-07220060 is from about 100 mg to about 500 mg BID.

Embodiment 8. The method of embodiment 7, wherein the endocrine therapy agent is an aromatase inhibitor, an androgen receptor inhibitor, a selective estrogen receptor degrader (SERD), or a selective estrogen receptor modulator (SERM).

Embodiment 9. The method of embodiment 7, wherein the endocrine therapy agent is selected from the group consisting of letrozole, anastrozole, exemestane, fulvestrant, elacestrant, amcenestrant, giredestrant, RG6171, camizestrant, AZD9496, rintodestrant, ZN-c5, LSZ102, D-0502, LY3484356, SHR9549, tamoxifen, raloxifene, toremifene, lasofoxifene, bazedoxifene and afimoxifene.

Embodiment 10. The method of embodiment 7, wherein the endocrine therapy agent is letrozole or fulvestrant.

Embodiment 11. The method of any one of embodiments 7-10, wherein the subject is administered the endocrine therapy agent and subsequently administered PF-07220060.

Embodiment 12. The method of embodiments 1 or 7, wherein the subject has been previously treated with chemotherapy, radiotherapy, and/or surgical resection.

Embodiment 13. The method of embodiments 1 or 7, wherein the subject has been previously treated with a CDK4/6 inhibitor.

Embodiment 14. The method of embodiments 1 or 7, wherein the subject has been previously treated with an endocrine therapy agent.

Embodiment 15. The method of embodiments 1 or 13, wherein the subject is a mammal.

Embodiment 16. The method of any one of embodiments 1 to 15, wherein the subject is suffering from breast cancer, ovarian cancer, fallopian tube cancer, primary peritoneal cancer, bladder cancer, uterine cancer, prostate cancer, lung cancer, esophageal cancer, head and neck cancer, colorectal cancer, kidney cancer, liver cancer, pancreatic cancer, stomach cancer, and thyroid cancer.

Embodiment 17. The method of any one of embodiments 1 to 16, wherein the cancer is breast cancer selected from any one or more of: hormone receptor positive (HR+), hormone receptor negative (HR−), human epidermal growth factor receptor 2 negative (HER2−), human epidermal growth factor receptor 2 positive (HER2+), HR+/HER2−, ER−/HR+, ER+/HER2−, and triple negative breast cancer (TNBC).

Embodiment 18. The method of any one of embodiments 1 to 16, wherein the cancer is NSCLC, prostate, colorectal cancer, liposarcoma, or tumors characterized by amplification or overexpression of CDK4 and/or CCND1.

Embodiment 19. Use of PF-07220060 in the manufacture of a medicament for use in the treatment of cancer in a subject in need thereof which the medicament is administered in a therapeutically effective unit dosage of PF-07220060 in the amount of from about 100 mg to about 500 mg BID.

Embodiment 20. Use of PF-07220060 and an endocrine therapy agent in the manufacture of a medicament for use in the treatment of cancer in a subject in need thereof which the medicament is administered in a therapeutically effective unit dosage of PF-07220060 in the amount of from about 100 mg to about 500 mg BID.

Embodiment 21. A medicament comprising a therapeutically effective amount of PF-07220060 for use in treating cancer in a subject in need thereof, wherein the therapeutically effective amount is from about 100 mg to about 500 mg BID.

Embodiment 22. A medicament comprising a therapeutically effective amount of PF-07220060 and an endocrine therapy agent for use in treating cancer in a subject in need thereof, wherein the therapeutically effective amount is from about 100 mg to about 500 mg BID.

### EXAMPLES

The following examples are merely illustrative of the disclosure and should not be considered limiting the scope of the invention in any way, as these examples and other equivalents thereof will become apparent to those skilled in the art in light of the present disclosure and the accompanying claims.

### Example 1

Multicellular Tumor Spheroid Growth Inhibition by Single Agent PF-07220060 and in Combination with Fulvestrant

The purpose of this example was to evaluate the combination effect of PF-07220060 and endocrine therapy in multicellular tumor spheroid growth inhibition (SGI). To model the characteristics and cellular behavior of human tumors, the activity of PF-07220060 was evaluated in three dimensional MCTS models of HR+ breast cancer and AR+ prostate cancer following PF-07220060 treatment as a single agent and in combination with fulvestrant
1) in HR+, HER2-T47D breast cancer spheroids.
2) in HR+, HER2+BT474 breast cancer spheroids,
3) in BT474 spheroids, and
4) in AR+ prostate carcinoma cell line (LNCaP) prostate cancer spheroids.
   Spheroids were treated with increasing concentrations of 100, 300, or 1000 nM PF-07220060 and compared to palbociclib at the clinically relevant concentration of 30 nM. Single agent PF-07220060 inhibited spheroid growth in a dose-dependent manner, with highest efficacy (86% SGI over the duration of treatment with 1000 nM) shown in the T47D BC spheroid model (Table 1). Combining 100 nM PF-07220060 with 1 nM fulvestrant in T47D cells increased SGI to 96% compared to either single agent alone (57 to 67% SGI with 100 nM PF-07220060 or 79% SGI with fulvestrant) (Table 1). BT474 spheroids were relatively less sensitive to PF-07220060 resulting in 52% SGI at 1000 nM.

PF-07220060 showed dose-dependent inhibition of LNCaP prostate cancer spheroid growth reaching 53% SGI at 1000 nM (Table 1).

In summary, these data support the use of PF-07220060 in combination with endocrine therapy in HR+, HER2− breast cancer. There is additive or synergistic benefit from the PF-07220060 combinations across multiple tumor types. When used as a single agent, PF-07220060 inhibits spheroid growth in a dose-dependent manner, with highest activity seen in the HR+, HER2− breast cancer spheroid model.

TABLE 1
Multicellular Tumor Spheroid Growth Inhibition by PF-07220060
Single Agent and in Combination with Fulvestrant

| | Single Agent PF-07220060 | | Combination Treatment | |
|---|---|---|---|---|
| | | SGI | | SGI |
| | Treatment | (%) | Treatment | (%) |
| T47D | 100 nM PF-07220060 | 57; 67 | 300 nM | 96 |
| HR+, | 300 nM PF-07220060 | 74 | PF-07220060 + | |
| HER2− | 1000 nM PF-07220060 | 86 | 1 nM Fulvestrant | |
| Breast | 30 nM Palbociclib | 38; 49 | 30 nM Palbociclib + | 96 |
| | 1 nM Fulvestrant | 79 | 1 nM Fulvestrant | |
| BT474 | 100 nM PF-07220060 | 9 | | |
| HR+, | 300 nM PF-07220060 | 16; 26; 20 | | |
| HER2− | 1000 nM PF-07220060 | 52 | | |
| Breast | 30 nM Palbociclib | 0; 5; 1 | | |
| | 100 nM Fulvestrant | 10 | | |
| LNCaP | 100 nM PF-07220060 | 27 | | |
| AR+ | 300 nM PF-07220060 | 38; 38 | | |
| Prostate | 1000 nM PF-07220060 | 53 | | |
| | 30 nM Palbociclib | 5 | | |
| | 100 nM Palbociclib | 24 | | |

SGI = (1-AUC treatment/AUC DMSO) × 100%. SGI for all treatment arms was derived at the timepoint when the vehicle (0.01% DMSO) treated spheroids reach their maximal diameter

### Example 2: Clinical Trial Protocol

An ongoing open-label, nonrandomized, dose-finding Phase 1/1b Study investigating the safety, tolerability, PK, and PD of PF-07220060 administered as a single agent and when administered in combination with endocrine therapy.

### Study Design

In Part 1A, single escalating doses of PF-07220060 alone will be administered beginning at 100 mg BID dose on a continuous basis to determine the maximum tolerated dose (MTD) and/or select the recommended phase 2 dose (RP2D) of PF-07220060 as monotherapy.

In Part 1B and Part 1C, PF-07220060 will be administered in combination with an endocrine therapy (letrozole or fulvestrant) to identify the MTD and/or select the RP2D of PF-07220060 with each endocrine agent.

Part 1D will assess the effect of food on PK of PF-07220060 at or below the monotherapy MTD. In Part 1D, participants will receive PF-07220060 as a single agent at or below the monotherapy MTD on Day-7 and then on a continuous basis starting on Day 1 to assess food effect.

Part 1E will assess the potential drug-drug-interaction (DDI) between PF-07220060 given at or below the monotherapy MTD established in Part 1A and midazolam.

Part 2B is an expansion cohort for combination therapy of PF-07220060 with letrozole in patients with HR-positive/HER2-negative advanced or metastatic breast cancer (mBC) who have not received any prior systemic anti-cancer therapies for their advanced disease.

Part 2C is an expansion cohort for combination therapy of PF-07220060 with fulvestrant (with or without goserelin) in patients with HR-positive/HER2-negative advanced or mBC whose disease has progressed on prior therapy.

Study entry is defined as Day 1 of dosing. All cycles are 28 days in length. The evaluation of an alternative dosing regimen (e.g., QD) may be considered during the course of dose escalation or after determination of the MTD/RP2D for the BID regimen based on emerging and available preliminary clinical data, including safety/tolerability, laboratory, PK and PD findings. Intermittently administered PF-07220060 dosing schedules (e.g., 3 weeks on, 1 week off) may also be evaluated if indicated based on emerging clinical data.

Participants experiencing toxicity including a DLT may be managed with dose modification or discontinuation from treatment. The proposed doses, schedule(s) and PK time points may change during the study based on the emerging safety and PK data.

The time on study can vary depending on the observed toxicity and potential benefit an individual participant derives. It is estimated that each participant may remain on treatment for approximately 6 to 8 cycles, making the total study duration approximately 24 to 32 weeks. Actual duration can be longer, if a participant derives benefit from study treatment.

Based on evolving PK, PD, and safety profiles assessed in Part 1A, the starting dose of PF-07220060 in Part 1B and Part 1C may commence at MTD or before reaching the MTD at a dose level determined to be safe by Bayesian Logistic Regression Model (BLRM) fulfilling the escalation with overdose control (EWOC) criteria in Part 1A. (Rogatko, A., et.al., Translation of Innovative Designs Into Phase Trials, J. Clin. Oncol., 25 (31), 4982-6, 2007). The use of the EWOC principle limits the risk of treating participants at a dose above the MTD. In addition, more than one dosing regimen (e.g., a different dose, dosing frequency) of PF-07220060 may be investigated in Part 1B and Part 1C.

### Study Population

The eligibility criteria are designed to select participants for whom participation in the study is considered appropriate. Inclusion criteria for patient selection include the following:

All participants—histologically or cytologically proven locally advanced or metastatic solid tumor, which is unrespectable or cannot be radiated with curative intent.

### Disease Requirements for Part 1

Part 1B and Part 1C:
Refractory HR-positive/HER2-negative (2L+ with prior CDK4/6) breast cancer.

Part 1A, Part 1D and Part 1E:
Refractory HR-positive/HER2-positive breast cancer.
Tumors other than breast cancer: NSCLC, prostate, CRC, liposarcoma, or tumors with previously confirmed CDK4 or CCND1 amplification according to local standard tests.

### Disease Requirements for Part 2

Part 2B and Part 2C:
HR-positive/HER2-negative breast cancer
Participants who are either:
   Postmenopausal women defined by at least one of the following criteria:
      Age≥60 years;
      Age<60 years and cessation of regular menses for at least 12 consecutive months with no alternative pathological or physiological cause; and serum estradiol and follicle-stimulating hormone (FSH) level within the laboratory's reference range for postmenopausal females;
      Documented bilateral oophorectomy;
      Medically confirmed ovarian failure.
Or
   For Part 2C: Pre/peri-menopausal, i.e., not meeting the criteria for being postmenopausal.
      Pre/perimenopausal women can be enrolled if amenable to be treated with the luteinizing hormone-releasing hormone (LHRH) agonist goserelin. Participants must have commenced treated with goserelin or an alternative LHRH agonist at least 4 weeks prior to enrollment. If participants have received an alternative LHRH agonist prior to study entry, it is preferable to switch to goserelin for the duration of the trial. However other LHRH antagonists, such as leuprolide is acceptable.

### Lesion Requirements:

For Part 1: participant must have evaluable lesion (including skin or bone lesion only).
For Part 2B and Part 2C: participants must have measurable disease as defined per RECIST (Response Evaluation Criteria in Solid Tumors) version 1.1 (Eisenhauer et al., European Journal of Cancer, 2009, 45 (2): 228-47). Tumor lesions previously irradiated or subjected to locoregional therapy will only be deemed measurable if progression at the treated site after completion of therapy is clearly documented.

### Prior Systemic Treatment:

For Part 1:
HR-positive/HER2-negative Breast Cancer (Part 1A/Part 1B/Part 1C/Part 1D/Part 1E). Participants should have received:
   at least 1 line of standard of care (SOC), including CDK4/6 inhibitor therapy for advanced or metastatic disease, or if CDK4/6 inhibitors are available but not considered appropriate in the opinion of the investigator, participants may be enrolled if a compelling clinical rationale is provided by the investigator and approved by the sponsor; or
   at least 1 line of anti-endocrine therapy in countries without CDK4/6 inhibitor approval or reimbursement, for advanced or metastatic disease.

Prior chemotherapies for advanced disease setting are allowed in both cases.
HR-positive/HER2-positive breast cancer (Part 1A/Part 1D): Participants should have received at least one prior treatment of approved HER2 targeting therapy.
Tumors other than breast cancer (Part 1A/Part 1D): tumor that is resistant to at least 2 lines of standard systemic therapy for advanced or recurrent disease or for which no standard therapy is available.

### For Part 2:

Part 2B: Participants who have not received any prior systemic anti-cancer therapies for advanced/metastatic breast cancer.

Part 2C:
Progressed during treatment or within 12 months of completion of adjuvant therapy with an aromatase inhibitor if postmenopausal, or tamoxifen if pre- or perimenopausal, or
Progressed while on or within 1 month after the end of prior aromatase inhibitor therapy for advanced/metastatic breast cancer if postmenopausal, or prior endocrine treatment for advanced/metastatic breast cancer if pre- or perimenopausal.
One previous line of chemotherapy for advanced/metastatic disease is allowed in addition to endocrine therapy.

### Inclusion Criteria

The inclusion criteria for the two Parts of the study are as follows:
1. Participants in both Part 1 and Part 2 (except for Part 2B) must be refractory to or intolerant of existing therapies known to provide clinical benefit for their condition.
2. Participants are ≥18 years.
3. Eastern Cooperative Oncology Group (ECOG) performance status is 0 or 1.
4. Adequate bone marrow function, as evidenced by:
   a. ANC≥1,500/mm³ or ≥1.5×10⁹/L;
   b. platelets≥100,000/mm³ or ≥100×10⁹/L;
   c. hemoglobin≥9 g/dL. Limited transfusions to reach this value are allowed, after discussion with sponsor's medical monitor. There should not be a chronic need for transfusions in the recent past (approximately 3 months).
5. Adequate renal function, defined as: estimated creatinine clearance≥50 mL/min for Part 1 as calculated using the method standard for the institution. In equivocal cases, a 24-hour urine collection test may be used to estimate the creatinine clearance more accurately.
6. Adequate liver function, as evidenced by:
   a. Total serum bilirubin≤1.5×ULN unless the participant has documented Gilbert syndrome (in which case, up to total serum bilirubin≤3.0×ULN will be allowed);
   b. AST and ALT≤2.5×ULN; ≤5.0×ULN if there is liver involvement by the tumor;
   c. ALKP≤2.5×ULN (≤5.0×ULN in case of bone or liver metastasis).

### Exclusion Criteria

Participants are excluded from the study if any of the following criteria apply:
1. For Part 1D: Participants who have had a gastrectomy or have dietary or other restrictions that preclude a 10 hour overnight fast (water permitted) or consumption of the high fat, high calorie meal.
2. For Part 2B: Prior neoadjuvant or adjuvant treatment with a non-steroidal aromatase inhibitor (i.e., anastrozole or letrozole) with disease recurrence while on or within 12 months of completing treatment, or prior treatment with any CDK4/6 inhibitor.
3. For Part 2C: prior treatment with any CDK inhibitor, or fulvestrant, or everolimus, or any agent whose mechanism of action is to inhibit the PI3K-mTOR pathway.
4. Known active uncontrolled or symptomatic CNS metastases, carcinomatous meningitis, or leptomeningeal disease as indicated by clinical symptoms, cerebral edema, and/or progressive growth. Participants with a history of CNS metastases or cord compression are eligible if they have been definitively treated (e.g., radiotherapy, stereotactic surgery) and are clinically stable off anticonvulsants and steroids for at least 4 weeks before enrollment and have no evidence of progression at time of study enrollment.
5. Participants with advanced/metastatic, symptomatic, visceral spread, that are at risk of life-threatening complications in the short term (including participants with massive uncontrolled effusions [pleural, pericardial, peritoneal], pulmonary lymphangitis, and over 50% liver involvement).
6. Any other active malignancy within 3 years prior to enrollment, except for adequately treated basal cell or squamous cell skin cancer, or carcinoma in situ.
7. Major surgery within 4 weeks prior to study entry.
8. Radiation therapy with treatment intent within 4 weeks prior to study entry. Any palliative radiation therapy must be completed within the 7 days prior to Day 1 of study intervention administration.
9. Last anticancer treatment within 2 weeks (or 5 half-lives, whichever is shorter), unless the last immediate anticancer treatment contained an antibody based agent(s) (approved or investigational), then the interval of 4 weeks (or 5 half-lives whichever is shorter) is required prior to receiving the study intervention.
10. For participants in Part 1C who received fulvestrant as part of the last prior therapy are eligible.
11. Previous high-dose chemotherapy requiring stem cell rescue.
12. Active and clinically significant bacterial, fungal, or viral infection, including but not limited to HBV, HCV, known HIV or AIDS-related illness.
13. Baseline 12-lead ECG that demonstrates clinically relevant abnormalities that may affect participant safety or interpretation of study results (e.g., baseline QTc interval >470 msec, complete LBBB, signs of an acute or indeterminate-age myocardial infarction, ST-T interval changes suggestive of active myocardial ischemia, second- or third-degree AV block, or serious bradyarrhythmias or tachyarrhythmias). If the baseline uncorrected QT interval is >470 msec, this interval should be rate-corrected using the Fridericia method and the resulting QTcF should be used for decision making and reporting. If QTc exceeds 470 msec, or QRS exceeds 120 msec, the ECG should be repeated 2 more times and the average of the 3 QTc or QRS values should be used to determine the participant's eligibility. Computer-interpreted ECGs should be overread by a physician experienced in reading ECGs before excluding participants. Cases must be discussed in detail with sponsor's medical monitor to judge eligibility.
14. Any of the following in the previous 6 months: myocardial infarction, congenital long QT syndrome, Torsades de pointes, arrhythmias (including sustained ventricular tachyarrhythmia and ventricular fibrillation), serious conduction system abnormalities (e.g., bifascicular block (defined as right bundle branch and let anterior or posterior hemiblock), 3rd degree AV block), unstable angina, coronary/peripheral artery bypass graft, symptomatic CHF, New York Heart Association class Ill or IV, cerebrovascular accident, transient ischemic attack, or symptomatic pulmonary embolism; deep venous thrombosis; arterial occlusive disease; ongoing cardiac dysrhythmias of NCI CTCAE Grade ≥2, atrial fibrillation of any grade that is uncontrolled, or QTcF interval >470 msec at screening.
15. Blood pressure (≥150/100 mmHg despite optimal medical therapy) that cannot be controlled.
16. Therapeutic dose of anti-coagulant treatment is prohibited (prophylactic doses of anticoagulant are allowed).
17. Known abnormalities in coagulation such as bleeding diathesis.
18. Known or suspected hypersensitivity to active ingredient/excipients of PF-07220060, letrozole, fulvestrant, enzalutamide and/or goserelin (or equivalent agent to induce chemical menopause/chemical castration).
19. Active inflammatory GI disease, known diverticular disease or previous gastric resection or lap band surgery. Impairment of GI function or GI disease that may significantly alter the absorption of PF-07220060, such as history of GI surgery which may result in intestinal blind loops and clinically significant gastroparesis, short bowel syndrome, unresolved nausea, vomiting, active inflammatory bowel disease or diarrhea of CTCAE Grade >1.
20. Current use of drugs which have a risk for QTc prolongation.
21. Treatment with strong and moderate CYP3A4/5 or UGT2B7 inhibitors, including their administration within 5 half-lives of the CYP3A4/5 or UGT2B7 inhibitor, whichever is longer, prior to first dose of study intervention.
22. Treatment with proton-pump inhibitors (e.g., dexlansoprazole, esmerpraole, lansoprazole, omeprazole, pantoprazole, rabeprazole) within 7 days prior to first dose of study intervention.
23. Other medical or psychiatric condition including recent (within the past year) or active suicidal ideation/behavior or laboratory abnormality that may increase the risk of study participation or, in the investigator's judgment, make the participant inappropriate for the study.
24. Pregnant or breast-feeding.

### Example 3: Clinical Trial results

The Phase 1/1b PF-07220060 clinical trial is ongoing. As of Sep. 2, 2022, a total of 66 participants (43 females and 23 males) have been treated with PF-07220060 in Part 1A, Part 1B, Part 1C and Part 1D. The mean age is 61.4 years (range 36-82) and the population includes participants with HR+ HER2− advanced or mBC, and other tumors that potentially thrive in a CDK4-dependent fashion, including adenocarcinoma of NSCLC, prostate cancer, CRC, liposarcoma, and tumors with previously confirmed CDK4 or CCND1 amplification according to local standard tests. The demographic of these 66 participants are provided in Table 2.

TABLE 2
Demographic Characteristics Categorized by Treatment Groups

| | | | | | Part 1B | | Part 1C | | | |
|---|---|---|---|---|---|---|---|---|---|---|
| Part 1A | | | | | 300 mg | 400 mg | 300 mg | 400 mg | Part 1D | |
| 100 mg | 200 mg | 300 mg | 400 mg | 500 mg | BID + | BID + | BID + | BID + | 400 mg | |
| BID | BID | BID | BID | BID | Letrozole | Letrozole | Fulvestrant | Fulvestrant | BID | Total |
| (N = 3) | (N = 8) | (N = 10) | (N = 9) | (N = 4) | (N = 6) | (N = 7) | (N = 6) | (N = 7) | (N = 6) | (N = 66) |

AGE (YEARS), N (%)

| | | | | | | | | | | | |
|---|---|---|---|---|---|---|---|---|---|---|---|
| <18 | 0 | 0 | 0 | 0 | 0 | 0 | 0 | 0 | 0 | 0 | 0 |
| 18-44 | 0 | 1 | 0 | 1 | 0 | 1 | 0 | 2 | 0 | 1 | 6 |
| | | (12.5%) | | (11.1%) | | (16.7%) | | (33.3%) | | (16.7%) | (9.1%) |
| 45-64 | 1 | 2 | 4 | 4 | 1 | 3 | 5 | 2 | 3 | 4 | 29 |
| | (33.3%) | (25.0%) | (40.0%) | (44.4%) | (25.0%) | (50.0%) | (71.4%) | (33.3%) | (42.9%) | (66.7%) | (43.9%) |
| ≥65 | 2 | 5 | 6 | 4 | 3 | 2 | 2 | 2 | 4 | 1 | 31 |
| | (66.7%) | (62.5%) | (60.0%) | (44.4%) | (75.0%) | (33.3%) | (28.6%) | (33.3%) | (57.1%) | (16.7%) | (47.0%) |
| Mean | 66.7 | 63.0 | 62.1 | 59.1 | 66.5 | 62.0 | 60.0 | 56.7 | 66.0 | 56.2 | 61.4 |
| (SD) | (8.74) | (13.37) | (8.72) | (12.10) | (5.07) | (10.95) | (12.60) | (12.61) | (6.19) | (10.05) | (10.48) |
| Median | 69.0 | 67.0 | 65.0 | 63.0 | 68.5 | 62.5 | 55.0 | 61.0 | 68.0 | 58.0 | 62.5 |
| (range) | (57, 74) | (36, 78) | (48, 74) | (41, 75) | (59, 70) | (44, 75) | (47, 82) | (41, 70) | (58, 73) | (41, 69) | (36, 82) |

GENDER

| | | | | | | | | | | | |
|---|---|---|---|---|---|---|---|---|---|---|---|
| Male | 2 | 4 | 7 | 3 | 3 | 0 | 0 | 0 | 0 | 4 | 23 |
| | (66.7%) | (50.0%) | (70%) | (33.3%) | (75%) | | | | | (66.7%) | (34.8%) |
| Female | 1 | 4 | 3 | 6 | 1 | 6 | 7 | 6 | 7 | 2 | 43 |
| | (33.3%) | (50.0%) | (30%) | (66.7%) | (25%) | (100%) | (100%) | (100%) | (100%) | (33.3%) | (65.2%) |

RACE

| | | | | | | | | | | | |
|---|---|---|---|---|---|---|---|---|---|---|---|
| White | 3 | 3 | 8 | 9 | 3 | 6 | 2 | 4 | 3 | 2 | 43 |
| | (100%) | (37.5%) | (80%) | (100%) | (75%) | (100%) | (28.6%) | (66.7%) | (42.9%) | (33.3%) | (65.2%) |
| Black or | 0 | 1 | 0 | 0 | 0 | 0 | 0 | 0 | 1 | 0 | 2 |
| African | | (12.5%) | | | | | | | (14.3%) | | (3.0%) |
| American | | | | | | | | | | | |
| Asian | 0 | 3 | 0 | 0 | 0 | 0 | 0 | 1 | 0 | 0 | 4 |
| | | (37.5%) | | | | | | (16.7%) | | | (6.1%) |
| Not | 0 | 1 | 2 | 0 | 1 | 0 | 5 | 1 | 3 | 4 | 17 |
| reported | | (12.5%) | (20%) | | (25%) | | (71.4%) | (16.7%) | (42.9%) | (66.7%) | (25.8%) |

ETHNICITY

| | | | | | | | | | | | |
|---|---|---|---|---|---|---|---|---|---|---|---|
| Hispanic | 0 | 1 | 1 | 1 | 1 | 0 | 0 | 0 | 0 | 0 | 4 |
| or Latino | | (12.5%) | (10%) | (11.1%) | (25%) | | | | | | (6.1%) |
| Not | 3 | 6 | 7 | 8 | 2 | 6 | 2 | 5 | 3 | 1 | 43 |
| Hispanic | (100%) | (75.0%) | (70%) | (88.9%) | (50%) | (100%) | (28.6%) | (83.3%) | (42.9%) | (16.7%) | (65.2%) |
| or Latino | | | | | | | | | | | |
| Not | 0 | 1 | 2 | 0 | 1 | 0 | 5 | 1 | 4 | 5 | 19 |
| reported | | (12.5%) | (20%) | | (25%) | | (71.4%) | (16.7%) | (57.1%) | (83.3%) | (28.8%) |

TUMOR TYPE

| | | | | | | | | | | | |
|---|---|---|---|---|---|---|---|---|---|---|---|
| Breast | 1 | 1 | 1 | 3 | 1 | 6 | 7 | 6 | 7 | 1 | 34 |
| Cancer | (33.3%) | (12.5%) | (10%) | (33.3%) | (25%) | (100%) | (100%) | (100%) | (100%) | (16.7%) | (51.5%) |
| Colorectal | 0 | 1 | 2 | 1 | 0 | 0 | 0 | 0 | 0 | 0 | 4 |
| Cancer | | (12.5%) | (20%) | (11.1%) | | | | | | | (6.1%) |
| Endometrial | 0 | 0 | 1 | 0 | 0 | 0 | 0 | 0 | 0 | 0 | 1 |
| cancer | | | (10%) | | | | | | | | (1.5%) |
| HNSCC | 0 | 0 | 1 | 0 | 0 | 0 | 0 | 0 | 0 | 0 | 1 |
| | | | (10%) | | | | | | | | (1.5%) |
| Liposarcoma | 0 | 1 | 0 | 1 | 1 | 0 | 0 | 0 | 0 | 2 | 5 |
| | | (12.5%) | | (11.1%) | (25%) | | | | | (33.3%) | (7.6%) |
| Melanoma | 0 | 0 | 0 | 0 | 0 | 0 | 0 | 0 | 0 | 1 | 1 |
| | | | | | | | | | | (16.7%) | (1.5%) |
| Non-small | 0 | 0 | 0 | 1 | 0 | 0 | 0 | 0 | 0 | 0 | 1 |
| cell lung | | | | (11.1%) | | | | | | | (1.5%) |
| cancer | | | | | | | | | | | |
| Prostate | 2 | 3 | 5 | 3 | 2 | 0 | 0 | 0 | 0 | 1 | 16 |
| Cancer | (66.7%) | (37.5%) | (50%) | (33.3%) | (50%) | | | | | (16.7%) | (24.2%) |
| Small | 0 | 1 | 0 | 0 | 0 | 0 | 0 | 0 | 0 | 0 | 1 |
| intestine | | (12.5%) | | | | | | | | | (1.5%) |
| cancer | | | | | | | | | | | |
| Squamous cell | 0 | 0 | 0 | 0 | 0 | 0 | 0 | 0 | 0 | 1 | 1 |
| carcinoma | | | | | | | | | | (16.7%) | (1.5%) |
| of lung | | | | | | | | | | | |
| Not reported | 0 | 1 | 0 | 0 | 0 | 0 | 0 | 0 | 0 | 0 | 1 |
| | | (12.5%) | | | | | | | | | (1.5%) |

N = number of participants

### Patient Population

A summary of the patient population in each part of the study is described in Table 3:

TABLE 3
Summary of Disease Type and Prior Treatments for Enrollment

| | | | |
|---|---|---|---|
| Part 1A/Part | | | |
| 1D/Part 1E | Part 1B & Part 1C | Part 2B | Part 2C |

Cancer Type

| | | | |
|---|---|---|---|
| Breast cancer, | HR-positive/HER2- | HR-positive/ | HR-positive/HER2-negative |
| NSCLC, prostate, | negative breast | HER2-negative | breast cancer |
| CRC, liposarcoma, | cancer | breast cancer | |
| or tumors with | | | |
| previously | | | |
| confirmed CDK4 or | | | |
| CCND1 | | | |
| amplification | | | |

Prior Systemic Treatment

| | | | |
|---|---|---|---|
| For HR- | At least 1 line of SOC, | No prior systemic | Progressed during treatment |
| positive/HER2- | including CDK4/6 | anti-cancer | or within 12 months of |
| negative BC: at | inhibitor therapy for | therapies for | completion of adjuvant therapy |
| least 1 line of SOC, | advanced or | advanced or | with an aromatase inhibitor if |
| including CDK4/6 | metastatic disease, or | metastatic breast | postmenopausal, or tamoxifen |
| inhibitor therapy, or | if CDK4/6 inhibitors | cancer. | if pre-or perimenopausal, or |
| at least 1 line of | are available but not | No prior | progressed while on or within |
| anti-ET in countries | considered | neoadjuvant or | 1 month after the end of prior |
| without CDK4/6 | appropriate in the | adjuvant | aromatase inhibitor therapy for |
| inhibitor approval. | opinion of the | treatment with a | advanced or metastatic breast |
| For HR- | investigator, | non-steroidal | cancer if postmenopausal, or |
| positive/HER2- | participants may be | aromatase | prior endocrine treatment for |
| positive BC: At | enrolled if a | inhibitor is | advanced/metastatic breast |
| least 1 prior | compelling clinical | allowed if the | cancer if pre- or |
| treatment of | rationale is provided | participant was | perimenopausal. |
| approved HER2 | by the investigator | with disease | No prior fulvestrant, CDK4/6, |
| targeting therapy. | and approved by the | recurrence while | mTOR or PI3K inhibitor. |
| For tumors other | sponsor, or at least 1 | on or within 12 | |
| than BC: resistant | line of anti-ET in | months of | |
| to at least 2 lines of | countries without | completing | |
| standard systemic | CDK4/6 inhibitor | treatment. | |
| therapy. | approval. | No prior CDK4/6 | |
| | | inhibitor. | |

### Part 1A-PF-07220060 Single Agent Dose Escalation

The purpose of Part 1A was to access the safety and tolerability of increasing doses of PF-07220060 as a single agent in sequential dose escalation cohorts in women of any menopausal status and men with HR-positive HER2-negative or HR-positive HER2-positive advanced or mBC, who progressed following at least one standard of care treatment (i.e., 2L and after) and participants with other solid tumors such as adenocarcinoma of NSCLC, prostate cancer, colorectal cancer, liposarcoma, and tumors with previously confirmed CDK4 or CCND1 amplification according to local standard tests. Patients with advanced or metastatic HR-positive mBC, under certain criteria, may receive the addition of endocrine therapy (either letrozole or fulvestrant) after at least two cycles of PF-07220060 monotherapy.

In Part 1A, single escalating doses of PF-07220060 alone was administered beginning at 100 mg BID dose on a continuous basis to determine the MTD and/or select the RP2D of PF-07220060 as monotherapy. Dosing will continue in sequential dose escalation cohorts until an MTD/RP2D can be estimated.

Thirty-four participants (n=34) have been treated in Part 1A (five dose cohorts) with 100 mg (n=3), 200 mg (n=8), 300 mg (n=10), 400 mg (n=9), and 500 mg (n=4) BID single agent PF-07220060.

Part 1B and Part 1C—PF-07220060 Dose Finding with Endocrine Therapy

The purpose of Part 1B and Part 1C was to determine the MTD/RP2D of PF-07220060 when given in combination with letrozole (Part 1B) or with fulvestrant (Part 1C), respectively, in women of any menopausal status and men with HR-positive HER2-negative advanced or mBC at 2L+ setting.

In Part 1B, participants received letrozole at the approved dose of 2.5 mg QD to be taken continuously together with PF-07220060. Thirteen participants (n=13) have been treated in Part 1B (two dose cohorts) with 300 mg (n=6) and 400 mg (n=7) BID PF-07220060 in combination with letrozole.

In Part 1C, participants received fulvestrant as two intramuscular (IM) injections (250 mg each) prior to receiving oral PF-07220060. Fulvestrant was administered on site by qualified medical personnel in Cycle 1, on Days 1 and 15, and on Day 1 (+1 to 5 days, depending on cycle number) of each subsequent 28-day cycle. Thirteen participants (n=13) have been treated in Part 1B (2 dose cohorts) with 300 mg (n=6) and 400 mg (n=7) BID PF-07220060 in combination with fulvestrant.

### Part 1D—PF-07220060 Food Effect

The purpose of Part 1D was to evaluate the effect of food on the pharmacokinetics (PK) of a single dose of PF-07220060 alone in participants.

Six participants (n=6) were treated with 400 mg BID single agent PF-07220060 in the morning under “fed conditions” on Cycle 1 Day-7 and under “fasted” conditions on Cycle 1 Day 1. For the evaluation of the food effect, PF-07220060 plasma concentration-time data of the participants was compared on Cycle 1 Day-7 to Cycle 1, Day 1. Natural log transformed AUC and Cmax values were analyzed using an analysis of variance model and treatment as fixed effects. Estimates of the mean differences (Fed-Fasted) and corresponding 90% CI were obtained from the model. The mean differences and 90% CI for the differences were exponentiated to provide estimates of the ratios of geometric means (Fed/Fasted) and 90% CI for the ratios.

### Part 1E—Midazolam Drug-Drug Interaction

The purpose of Part 1E is to assess the effect of repeated administration of PF-07220060 at the monotherapy MTD on the PK of oral midazolam in participants with advanced solid tumors. In Part 1E, Midazolam 2 mg is administered orally to participants on Cycle 1 Day (−1) alone and on Cycle 1 Day 15 together with PF-07220060. Only two doses of midazolam are given.

Part 2B and Part 2C-PF-07220060 Dose Expansion with Endocrine Therapy

The purpose of Part 2B is to evaluate the tolerability, safety, and preliminary anti-tumor activity of PF-07220060 in combination with letrozole. Part 2B is a dose expansion of PF-07220060 in combination with letrozole at the dose(s) identified in Part 1B in participants with HR-positive, HER2-negative advanced/metastatic breast cancer who have not received any prior systemic anti-cancer therapies for their advanced disease. In Part 2B, participants receive letrozole at the approved dose of 2.5 mg QD to be taken continuously together with PF-07220060.

The purpose of Part 2C is to evaluate the tolerability, safety, and preliminary anti-tumor activity of PF-07220060 in combination with fulvestrant. Part 2C is a dose expansion of PF-07220060 in combination with fulvestrant at the dose(s) identified in Part 1C in participants with HR-positive, HER2-negative advanced/metastatic breast cancer whose disease has progressed on prior therapy. Participants who have prior treatment with CDK4/6 inhibitors, fulvestrant, everolimus, and any agents whose MOA is to inhibit PI3k-mTOR pathway are excluded. In Part 2C, participants receive fulvestrant as two IM injections (250 mg each) prior to receiving oral PF-07220060.

To enable the optimal dosing regimen (e.g., different starting dose or different schedule) of PF-07220060 in combination with letrozole or fulvestrant to be identified, an additional dosing regimen(s) determined to be safe by BLRM in Part 1B and Part 1C may be investigated in Part 2B and Part 2C.

### Method of Administration:

### PF-07220060

Oral PF-07220060 was administered with at least 8 oz (240 mL) of water on an empty stomach (except during the food effect evaluation on Day-7 in Part 1D). No food or liquids other than water was consumed for 2 hours before and 1 hour following each dose throughout the study for BID dosing. No food or liquids other than water was consumed for 2 hours before and 2 hours after each dose throughout the study for QD dosing.

PF-07220060 was provided as tablets for oral administration, as the 5 mg, 25 mg, 100 mg, 125 mg, and 200 mg immediate release tablets.

For Part 1D only, PF-07220060 was administered following an overnight fast of at least 10 hours on Cycle 1, Day-7 and Cycle 1, Day 1. On Cycle 1, Day-7 (Fed state) a test breakfast meal (described below) was provided and must be consumed within 30 minutes. PF-07220060 was administered with approximately 8 oz of water 30 minutes after the start of the meal. No additional food was allowed until at least 4 hours post-dose. On Cycle 1, Day 1, participants received another single oral dose of PF-07220060 under fasted condition following an overnight fast of at least 10 hours. PF-07220060 was administered with 8 oz of water. No food was allowed for an additional 4 hours post-dose. For either treatment day, water was allowed ad libitum except for 1 hour before and 1 hour after drug administration. Starting from Cycle 1, Day 1 onwards, PF-07220060 was administered with at least 8 oz of water on an empty stomach, with no food or liquids other than water for 2 hours before and 2 hours following dosing.

The test breakfast meal to be consumed was a high fat (approximately 50% of total caloric content of the meal) and high calorie (approximately 800-1000 calories) meal. This test meal derived approximately 150, 250, and 500-600 calories from protein, carbohydrate, and fat, respectively.

### Fulvestrant

Fulvestrant (Faslodex®) was available as two 5-mL clear neutral glass (Type 1) barrels, each containing 250 mg/5 ml of fulvestrant solution for intramuscular injection and fitted with a tamper evident closure. The syringes are presented in a tray with polystyrene plunger rod and safety needles (SafetyGlide™) for connection to the barrel.

Fulvestrant injections were completed before PF-07220060 administration. Fulvestrant was administered intramuscularly in the gluteal area slowly (1-2 minutes per injection) as two 5 mL injections, per administration instructions provided in the Faslodex® label, during Cycle 1, on Days 1 and 15 and on Day 1 (+3 days) of each subsequent 28-day cycle.

Participants in Part 1C who were receiving fulvestrant as part of the last prior therapy would only require Day 1 dosing on Cycle 1 (i.e., Day 15 dosing was not required). The Cycle 1, Day 1 date occurred approximately 28 days from the last administration of fulvestrant.

### Letrozole

Letrozole (Femara®) was available as 2.5 mg tablets.

Letrozole was administered orally once daily (2.5 mg QD) continuously together with PF-07220060. Letrozole was not required to be given simultaneously with PF-07220060.

### Midazolam

Midazolam is a benzodiazepine used clinically for conscious sedation. It is specifically metabolized by CYP3A and is widely used as an in vivo probe for CYP3A activity. An oral formulation of midazolam is used to evaluate the effects on CYP3A activity in the GI tract and the liver. The resulting information will be used to determine whether any restrictions or dose modifications of concomitant medications are appropriate in future studies.

Participants are refrained from taking food and liquids except water in the 2-hour period prior to midazolam dosing and 1h after dosing. A 2 mg midazolam dose will be orally administered with 8 oz (240 mL) of water to participants for DDI assessment at Day-1 and Cycle 1 Day 15. Only two doses of midazolam will be given.

### I. Pharmacokinetics (Pk) Study

In the ongoing Phase 1/1b Study, as of Aug. 30, 2022, preliminary PK data for PF-07220060 as monotherapy (Part 1A) and in combination with endocrine therapy (Part 1B and Part 1C) was available from 60 participants with advanced solid tumors. PF-07220060 was administered orally as a single agent at doses ranging from 100 to 500 mg BID, and in combination with either letrozole or fulvestrant at 300 to 400 mg BID. Available plasma concentration-time data of PF-07220060 were analyzed using noncompartmental methods.

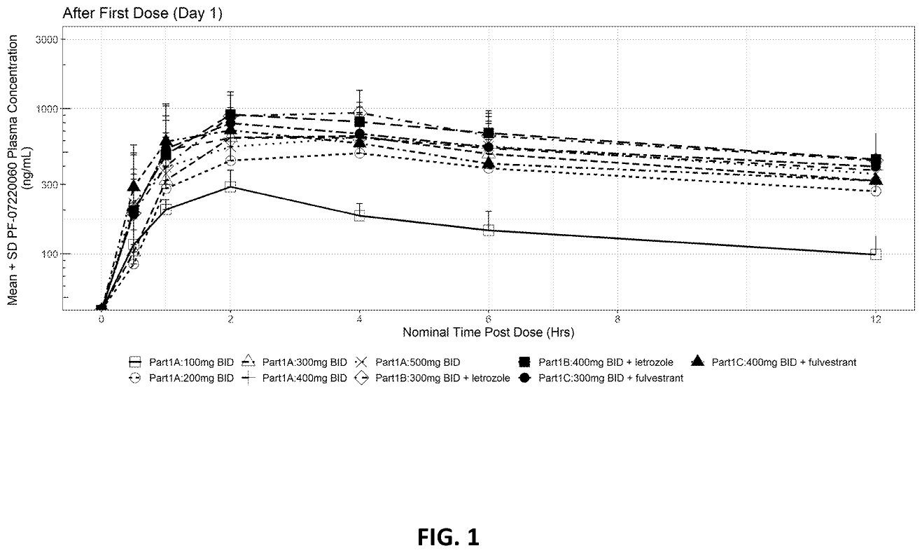

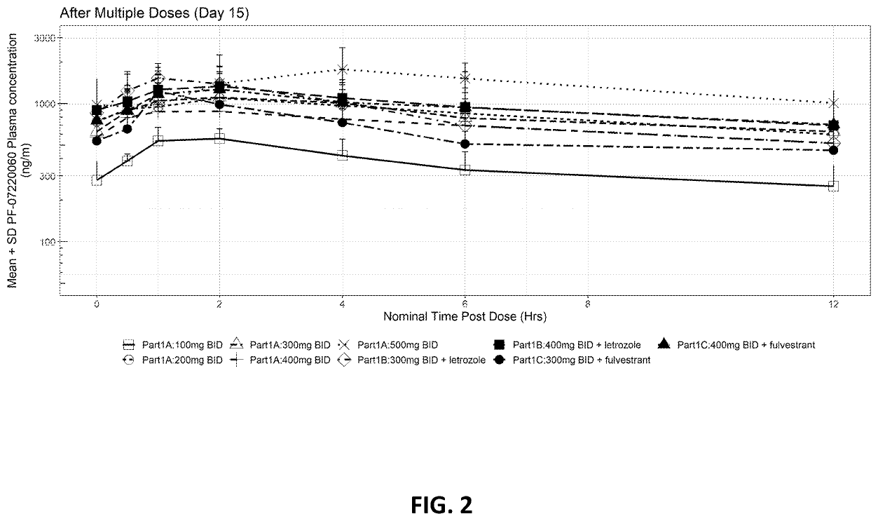

Preliminary noncompartmental PK analysis was conducted based on available samples collected from participants in the Study. FIG. 1 shows the plasma concentration-time profile of PF-07220060 on Day 1 following oral BID administration. FIG. 2 shows the plasma concentration-time profile of PF-07220060 on Day 15 following oral BID administration. Table 4 presents the summary of PK parameters for PF-07220060 as monotherapy, and in combination with letrozole or fulvestrant.

TABLE 4
Preliminary plasma Pharmacokinetic Parameters of PF-07220060 Following BID Oral Dose Administration
of PF-07220060 as Monotherapy and in Combination with Letrozole or Fulvestrant.

| | PF- | Post first dose (C1D1) | | | | Post multiple doses (C1D15) | | | | | | | |
|---|---|---|---|---|---|---|---|---|---|---|---|---|---|
| | 07220060 | | | | AUC₁₂ | | | | AUCₜₐᵤ | | | | |
| | Dose | | Cₘₐₓ | Tₘₐₓ | (ng · | | Cₘₐₓ | Tₘₐₓ | (ng · | Cₘᵢₙ | t_{1/2} | CL/F | R_{ac}, |
| Part | (mg, BID) | N | (ng/mL) | (hr) | hr/mL) | N | (ng/mL) | (hr) | hr/mL) | (ng/mL) | (hr) | (L/hr) | AUC |
| 1A | 100 | 3 | 280.2 | 2.0 | 1784 | 3 | 616.8 | 1.0 | 4063 | 231.4 | ND | 24.61 | 2.15 |
| | | | (30) | (1.9-2.0) | (22) | | (11) | (0.02-1.9) | (3095, 5336)^{b} | (40) | | (18.74, 32.32)^{b} | (1.97, 2.33)^{b} |
| | 200 | 8 | 454.4 | 3.9 | 2329 | 6 | 1010 | 2.5 | 6368 | 440 | 5.1 | 31.41 | 2.21 |
| | | | (77) | (1.1-10.2) | (77) | | (77) | (1.0-5.6) | (70) | (100) | | (70) | (0.28) |
| | 300 | 10 | 740.7 | 3.8 | 4564 | 9 | 1169 | 2.1 | 8095 | 470.4 | ND | 37.06 | 2.09 |
| | | | (24) | (1.9-4.1) | (24) | | (35) | (0.5-3.6) | (40) | (45) | | (40) | (0.98) |
| | 400 | 9 | 765.8 | 2.1 | 5113 | 8 | 948.9 | 1.0 | 8003 | 450.3 | 6.6 | 49.98 | 1.88 |
| | | | (33) | (1.0-6.1) | (35) | | (40) | (0.5-5.4) | (47) | (45) | (6.2, 7.0)^{b} | (47) | (0.90) |
| | 500 | 4 | 637 | 1.9 | 4172 | 4 | 1758 | 4.0 | 12470^{a} | 848.8 | ND | 40.1^{a} | 2.99^{a} |
| | | | (59) | (1.0-4.0) | (80) | | (36) | (2.1-4.0) | | (31) | | | |
| 1B | 300 + | 6 | 988.6 | 3.0 | 6439 | 6 | 1525 | 1.0 | 10360 | 487.1 | 4.1 | 28.95 | 1.67 |
| | letrozole | | (38) | (1.0-4.0) | (45) | | (33) | (0.5-2.0) | (27) | (25) | (0.8) | (27) | (1.35, 2.0)^{b} |
| | 400 + | 7 | 950.3 | 2.2 | 7108 | 6 | 1425 | 1.6 | 11460 | 728.3 | ND | 34.9 | 1.87 |
| | letrozole | | (41) | (1.0-4.0) | (45) | | (35) | (1.0-2.0) | (28) | (26) | | (28) | (0.94) |
| 1C | 300 + | 6 | 849.4 | 2.0 | 6129 | 6 | 1189 | 1.0 | 7891 | 347.4 | 5.7 | 38.02 | 1.36 |
| | fulvestrant | | (20) | (1.0-4.0) | (9) | | (42) | (1.0-2.1) | (27) | (51) | (1.0) | (27) | (0.36) |
| | 400 + | 7 | 730.5 | 2.2 | 4843 | 7 | 1384 | 1.1 | 9556 | 563.3 | 6.6 | 41.86 | 2.29 |
| | fulvestrant | | (45) | (1.0-4.2) | (33) | | (24) | (1.0-6.2) | (15) | (38) | | (15) | (0.84) |

^{a}n = 1;
^{b}n = 2
Abbreviations: CV = coefficient of variation; N = number of participants in the treatment group evaluable for PK parameters; ND = not determined; n = number of participants with AUCₜₐᵤ, R_{ac}, t_{1/2}, CL/F determined; PK = pharmacokinetic; SD = standard deviation.
Note:
Geometric mean (geometric % CV) for all parameters except median (min-max) for Tₘₐₓ and arithmetic mean (SD) for T_{1/2} and R_{ac}; SD and % CV not reported when the number of subjects with available parameters is <3;
ND = not determined.

Preliminary PK results showed that PF-07220060 was rapidly absorbed following oral dose administration with median Tmax values of 1 to 4 hours. The exposure parameters of PF-07220060, including Cmax, A U C, and Cmin, were generally increased generally increased with dose from 100 to 300 mg BID for both Cycle 1 Day 1 and Cycle 1 Day 15. No further increase in exposure parameters was observed at doses higher than 300 mg BID. Moderate accumulation of the AUC was observed following repeated BID dosing. In general, PF-07220060 exhibited moderate inter-subject variability in exposure parameters. PK of PF-07220060 as monotherapy and in combination with letrozole or fulvestrant were generally comparable.

### II. Safety

### Dose Limiting Toxicities (DLTs)

As of Sep. 2, 2022, of the 55 evaluable participants, 4 (7.3%) participants experienced DLTs. Two participants in the single agent 500 mg BID PF-07220060 cohort experienced DLTs of Grade 3 thrombocytopenia. One participant in the single agent 200 mg BID PF-07220060 cohort experienced DLT of Grade 3 neutropenia, and one participant in the combination 400 mg BID PF-07220060 with fulvestrant cohort experienced DLT of Grade 3 neutropenia≥5 days.

### Adverse Events

Adverse events of the patients dosed with PF-07220060 in the Phase 1/1b clinical trial were evaluated using the National Cancer Institute Common Terminology Criteria for Adverse Events (NCI CTCAE) version 4.03. As of Sep. 2, 2022 the following adverse events data were obtained.

### All-Causality Treatment-Emergent Adverse Events (TEAE)

A total of 375 all-causality TEAEs were reported in 59 (89.4%) of the 66 participants dosed with PF-07220060 in Part 1A, Part 1B, Part 1C and Part 1D.

In Part 1A (single agent PF-07220060), a total of 32 (94.1%) of the 34 participants reported all-causality TEAEs. Most frequently reported (≥20%) all-causality TEAEs across all dose levels were neutropenia (41.2%), anaemia, diarrhoea, leukopenia (35.3% each), nausea (29.4%), aspartate aminotransferase increased, and vomiting (20.6% each). Across all dose levels, a total of 15 Grade 3 or higher all-causality TEAEs were reported. Most frequently reported (≥5%) Grade 3 or higher all-causality TEAEs were neutropenia (14.7%), anaemia, aspartate aminotransferase increased, COVID-19, diarrhoea, leukopenia, thrombocytopenia, and syncope (5.9% each). No Grade 4 TEAEs were reported. One participant in the 400 mg BID group experienced a Grade 5 all-causality TEAE (respiratory failure; unrelated to study drug PF-07220060).

In Part 1B (PF-07220060 in combination with letrozole), 10 (76.9%) of the 13 participants reported all causality TEAEs. Most frequently reported (≥20%) all causality TEAEs were diarrhoea (38.5%), fatigue (30.8%), and nausea, neutropenia and sinusitis (23.1% each). Across all dose levels, one participant (7.7%) reported Grade 3 all-causality TEAEs including anaemia, leukopenia, and neutropenia (7.7% each). No Grade 4 and Grade 5 all causality TEAEs were reported.

In Part 1C (PF-07220060 in combination with fulvestrant), 13 out of 13 (100%) participants experienced at least 1 all causality TEAE. Most frequently reported (≥20%) all causality TEAEs were neutropenia (53.8%), diarrhoea, leukopenia and nausea (46.2% each), thrombocytopenia, and hyperglycaemia (30.8% each), anaemia, fatigue, and vomiting (23.1% each). Across all dose levels, a total of 7 Grade 3 all causality TEAEs were reported, which included neutropenia (23.1%), leukopenia (15.4%), and anaemia, nausea, and biliary tract infection (7.7% each). No Grade 4 or 5 TEAEs were reported.

In Part 1D (single agent PF-07220060, 400 mg BID food cohort), a total of 4 (66.7%) of the 6 participants reported TEAEs. Most frequently reported (≥20%) all causality TEAEs were diarrhoea, leukopenia, and neutropenia (50.0% each). No Grade 3 or 5 TEAEs were reported. One participant reported Grade 4 all causality TEAE (respiratory failure).

### Treatment-Related Treatment Emergent Adverse Events

Of the 66 participants dosed with PF-07220060 as single agent in Part 1 dose escalation, 54 (81.8%) participants reported any treatment-related TEAE. Overall, the most frequently reported (≥20%) treatment-related TEAEs were neutropenia (40.9%), diarrhea (33.3%), leukopenia (30.3%), nausea (27.3%), and anemia (21.2%).

In Part 1A (single agent PF-07220060), of the 34 participants 27 (79.4%) participants experienced at least one treatment-related TEAE.

The most frequently reported (≥10%) treatment-related TEAEs across all dose levels were neutropenia (41.2%), anemia and leukopenia (29.4% each), nausea (26.5%), diarrhoea (23.5%), thrombocytopenia (20.6%), vomiting (14.7%) and fatigue (11.8%). A total of 9 (26.5%) participants had Grade 3 treatment-related TEAEs. The most frequently reported (≥5%) Grade 3 treatment-related TEAEs were neutropenia (14.7%), aneamia, diarrhoea, leukopenia and thrombocytopenia (5.9% each). There were no Grade 4 or Grade 5 treatment-related TEAEs reported.

In Part 1B (PF-07220060 in combination with letrozole), a total of 10 (76.9%) of the 13 participants reported treatment-related TEAEs. Most frequently reported (≥20%) treatment-related TEAEs were diarrhoea (38.5%), fatigue (30.8%), nausea and neutropenia (23.1% each). One participant (7.7%) across cohorts reported Grade 3 treatment-related TEAEs, including anaemia, leukopenia, and neutropenia (7.7% each). No Grade 4 and Grade 5 TEAEs were reported.

In Part 1C (PF-07220060 in combination with fulvestrant), all 13 participants (100%) experienced at least one treatment related TEAE. Most frequently reported (≥20%) treatment-related TEAEs were neutropenia (53.8%), diarrhoea, and leukopenia (46.2% each), nausea (38.5%), hyperglycemia (30.8%), anaemia, fatigue, vomiting and thrombocytopenia (23.1% each). A total of 6 out of 13 (46.2%) participants had Grade 3 treatment-related TEAEs. The Grade 3 treatment-related TEAEs reported were neutropenia (23.1%), leukopenia (15.4%), and anaemia and nausea (7.7% each). No Grade 4 or 5 TEAEs were reported.

In Part 1D (single agent PF-07220060, 400 mg BID food cohort), a total of 4 (66.7%) of the 6 participants reported treatment-related TEAEs. Most frequently reported (≥20%) treatment-related TEAEs were diarrhoea, leukopenia, and neutropenia (50.0% each). No Grade 3, 4 nor 5 treatment-related TEAEs were reported.

### Safety Conclusion:

The available safety results support the ongoing clinical development of PF-07220060 for treatment of HR+, HER2− advanced or mBC and potentially other tumors that thrive in a CDK4-dependent fashion, including adenocarcinoma of NSCLC, prostate cancer, CRC, liposarcoma, and tumors with previously confirmed CDK4 or CCND1 amplification according to local standard tests.

### III. Efficacy

The efficacy data was obtained from the 70 evaluable patients. As of Oct. 15, 2022, PF-06873600 demonstrated a disease control rate of 65.7% (n=65.7/100) in evaluable patients across all cohorts. This included one participant with confirmed complete response, and six participants with confirmed partial responses. Among the evaluable patients who assessed as per RECIST version 1.1 (Table 5), the disease control rate was 57.9% (n=22/38) in the single agent PF-07220060 group (Total Part 1A), 80.8% (n=21/26) in the combination group PF-07220060+endocrine therapy (Part 1B+Part 1C).

TABLE 5
Best Overall Response

| | | | PF-07220060 in | Food effect on | |
|---|---|---|---|---|---|
| | | | Combination with | PK of PF-07220060 | |
| PF-07220060 | PF-07220060 + | PF-07220060 + | Endocrine Therapy | at or below | |
| Single Agent | letrozole | fulvestrant | Total Part | monotherapy MTD | |
| Total Part 1A | Total Part 1B | Total Part 1C | 1B + Part 1C | Part 1D | Total |
| (n = 38) | (n = 13) | (n = 13) | (n = 26) | (n = 6) | (n = 70) |

Confirmed Best
Overall Response,
n (%)

| Complete | 0 | | 1 | (7.7) | 0 | | 1 | (3.8) | 0 | | 1 | (1.4) |
|---|---|---|---|---|---|---|---|---|---|---|---|---|
| response (CR) | | | | | | | | | | | | |
| Partial | 0 | | 2 | (15.4) | 3 | (23.1) | 5 | (19.2) | 1 | (16.7) | 6 | (8.6) |
| response (PR) | | | | | | | | | | | | |
| Stable | 18 | (47.4) | 5 | (38.5) | 7 | (53.8) | 12 | (46.2) | 1 | (16.7) | 31 | (44.3) |
| disease (SD) | | | | | | | | | | | | |
| Progressive | 6 | (15.8) | 3 | (23.1) | 2 | (15.4) | 5 | (19.2) | 2 | (33.3) | 13 | (18.6) |
| disease (PD) | | | | | | | | | | | | |
| Non-CR/Non-PD | 4 | (10.5) | 2 | (15.4) | 1 | (7.7) | 3 | (11.5) | 1 | (16.7) | 8 | (11.4) |
| Not evaluable (NE) | 9 | (23.7) | 0 | | 0 | | 0 | | 1 | (16.7) | 10 | (14.3) |
| Reason for NE, n (%) | | | | | | | | | | | | |
| No adequate | 1 | (2.6) | 0 | | 0 | | 0 | | 1 | (16.7) | 2 | (2.9) |
| baseline assessment | | | | | | | | | | | | |
| No post-baseline | 7 | (18.4) | 0 | | 0 | | 0 | | 0 | | 7 | (10.0) |
| assessments due to | | | | | | | | | | | | |
| other reasons | | | | | | | | | | | | |
| SD too early [<7 | 1 | (2.6) | 0 | | 0 | | 0 | | 0 | | 1 | (1.4) |
| weeks after | | | | | | | | | | | | |
| Treatment Start | | | | | | | | | | | | |
| date] | | | | | | | | | | | | |
| Objective Response | 0 | | 3 | (23.1) | 3 | (23.1) | 6 | (23.1) | 1 | (16.7) | 7 | (10.0) |
| (CR + PR), n (%) | | | | | | | | | | | | |
| 95% CI | 0.0, 9.3 | | 5.0, 53.8 | | 5.0, 53.8 | | 9.0, 43.6 | | 0.4, 64.1 | | 4.1, 19.5 | |
| Number of | 30 | | 0 | | 0 | | 0 | | 5 | | 35 | |
| Patients without | | | | | | | | | | | | |
| Breast Cancer | | | | | | | | | | | | |
| Objective Response | 0 | | 0 | | 0 | | 0 | | 1 | (20.0) | 1 | (2.9) |
| (CR + PR) for | | | | | | | | | | | | |
| Patients without | | | | | | | | | | | | |
| Breast Cancer, n (%) | | | | | | | | | | | | |
| 95% CI | 0.0, 11.6 | | NE, NE | | NE, NE | | NE, NE | | 0.5, 71.6 | | 0.1, 14.9 | |
| Number of | 8 | | 13 | | 13 | | 26 | | 1 | | 35 | |
| Patients with | | | | | | | | | | | | |
| Breast Cancer | | | | | | | | | | | | |
| Objective Response | 0 | | 3 | (23.1) | 3 | (23.1) | 6 | (23.1) | 0 | | 6 | (17.1) |
| (CR + PR) for | | | | | | | | | | | | |
| Patients with Breast | | | | | | | | | | | | |
| Cancer, n (%) | | | | | | | | | | | | |
| 95% CI | 0.0, 36.9 | | 5.0, 53.8 | | 5.0, 53.8 | | 9.0, 43.6 | | 0.0, 97.5 | | 6.6, 33.6 | |
| Clinical Benefit | 2 | (25.0) | 4 | (30.8) | 8 | (61.5) | 12 | (46.2) | 0 | | 14 | (40.0) |
| Response, n (%) | | | | | | | | | | | | |
| 95% CI | 3.2, 65.1 | | 9.1, 61.4 | | 31.6, 86.1 | | 26.6, 66.6 | | 0.0, 97.5 | | 23.9, 57.9 | |
| Number of | 5 | | 10 | | 11 | | 21 | | 1 | | 27 | |
| Patients with | | | | | | | | | | | | |
| Breast Cancer | | | | | | | | | | | | |
| (with Measurable | | | | | | | | | | | | |
| Disease at Baseline) | | | | | | | | | | | | |
| Objective Response | 0 | | 3 | (30.0) | 3 | (27.3) | 6 | (28.6) | 0 | | 6 | (22.2) |
| (CR + PR) for | | | | | | | | | | | | |
| Patients with Breast | | | | | | | | | | | | |
| Cancer (with | | | | | | | | | | | | |
| Measurable Disease | | | | | | | | | | | | |
| at Baseline), n (%) | | | | | | | | | | | | |
| 95% CI | 0.0, 52.2 | | 6.7, 65.2 | | 6.0, 61.0 | | 11.3, 52.2 | | 0.0, 97.5 | | 8.6, 42.3 | |
| Clinical Benefit | 2 | (40.0) | 3 | (30.0) | 8 | (72.7) | 11 | (52.4) | 0 | | 13 | (48.1) |
| Response, n (%) | | | | | | | | | | | | |
| 95% CI | 5.3, 85.3 | | 6.7, 65.2 | | 39.0, 94.0 | | 29.8, 74.3 | | 0.0, 97.5 | | 28.7, 68.1 | |
| Number of | 6 | | 9 | | 11 | | 20 | | 1 | | 27 | |
| Patients with prior | | | | | | | | | | | | |
| CDK4/6 inhibitor | | | | | | | | | | | | |
| (with Measurable | | | | | | | | | | | | |
| Disease at Baseline) | | | | | | | | | | | | |
| Objective Response | 0 | | 3 | (33.3) | 3 | (27.3) | 6 | (30.0) | 0 | | 6 | (22.2) |
| (CR + PR) for | | | | | | | | | | | | |
| Patients with prior | | | | | | | | | | | | |
| CDK4/6 inhibitor (with | | | | | | | | | | | | |
| Measurable Disease | | | | | | | | | | | | |
| at Baseline), n (%) | | | | | | | | | | | | |
| 95% CI | 0.0, 45.9 | | 7.5, 70.1 | | 6.0, 61.0 | | 11.9, 54.3 | | 0.0, 97.5 | | 8.6, 42.3 | |
| Disease Control | 22 | (57.9) | 10 | (76.9) | 11 | (84.6) | 21 | (80.8) | 3 | (50.0) | 46 | (65.7) |
| (CR + PR + SD + Non- | | | | | | | | | | | | |
| CR/Non-PD), n (%) | | | | | | | | | | | | |
| 95% CI | 40.8, 73.7 | | 46.2, 95.0 | | 54.6, 98.1 | | 60.6, 93.4 | | 11.8, 88.2 | | 53.4, 76.7 | |
| Clinical Benefit | 10 | (26.3) | 4 | (30.8) | 8 | (61.5) | 12 | (46.2) | 2 | (33.3) | 24 | (34.3) |
| Response, n (%) | | | | | | | | | | | | |

| | | | | | | |
|---|---|---|---|---|---|---|
| 95% CI | 13.4, 43.1 | 9.1, 61.4 | 31.6, 86.1 | 26.6, 66.6 | 4.3, 77.7 | 23.3, 46.6 |

n = number of participants

PF-07220060 has demonstrated early signs of anti-tumor activity in combination with endocrine therapy in HR+/Her2-metastatic breast cancer patient population. Efficacy evaluation in dose expansion cohorts at RDE in combination with fulvestrant and letrozole is ongoing.

1. A method of treating cancer comprising orally administering to a subject in need thereof a therapeutically effective amount of PF-07220060, or a pharmaceutically acceptable salt thereof, wherein the therapeutically effective amount is from about 100 mg to about 500 mg twice per day (BID).

2. The method of claim 1, wherein the therapeutically effective amount is from about 300 mg to about 500 mg BID.

3. The method of claim 1, wherein the therapeutically effective amount is about 300 mg BID.

4. The method of claim 1, wherein the therapeutically effective amount is about 400 mg BID.

5. The method of any one of claim 1, wherein PF-07220060 is administered continuously.

6. The method of any one of claim 1, wherein PF-07220060 is administered in a tablet or capsule form.

7. A method of treating cancer comprising administering to a subject in need thereof a therapeutically effective amount of a pharmaceutical composition comprising PF-07220060 and an endocrine therapy agent to a subject in need thereof, wherein the therapeutically effective amount of PF-07220060 is from about 100 mg to about 500 mg BID.

8. The method of claim 7, wherein the endocrine therapy agent is an aromatase inhibitor, an androgen receptor inhibitor, a selective estrogen receptor degrader (SERD), or a selective estrogen receptor modulator (SERM).

9. The method of claim 7, wherein the endocrine therapy agent is selected from the group consisting of letrozole, anastrozole, exemestane, fulvestrant, elacestrant, amcenestrant, giredestrant, RG6171, camizestrant, AZD9496, rintodestrant, ZN-c5, LSZ102, D-0502, LY3484356, SHR9549, tamoxifen, raloxifene, toremifene, lasofoxifene, bazedoxifene and afimoxifene.

10. The method of claim 7, wherein the endocrine therapy agent is letrozole or fulvestrant.

11. The method of claim 7, wherein the subject is administered the endocrine therapy agent and subsequently administered PF-07220060.

12. The method of claim 1, wherein the subject has been previously treated with chemotherapy, radiotherapy, and/or surgical resection.

13. The method of claim 1, wherein the subject has been previously treated with a CDK4/6 inhibitor.

14. The method of claim 1, wherein the subject has been previously treated with an endocrine therapy agent.

15. The method of claim 1, wherein the subject is a mammal.

16. The method of claim 1, wherein the subject is suffering from breast cancer, ovarian cancer, fallopian tube cancer, primary peritoneal cancer, bladder cancer, uterine cancer, prostate cancer, lung cancer, esophageal cancer, head and neck cancer, colorectal cancer, kidney cancer, liver cancer, pancreatic cancer, stomach cancer, and thyroid cancer.

17. The method of claim 1, wherein the cancer is breast cancer selected from any one or more of: hormone receptor positive (HR+), hormone receptor negative (HR−), human epidermal growth factor receptor 2 negative (HER2−), human epidermal growth factor receptor 2 positive (HER2+), HR+/HER2−, ER−/HR+, ER+/HER2− and triple negative breast cancer (TNBC).

18. The method of claim 1, wherein the cancer is NSCLC, prostate, colorectal cancer, liposarcoma, or tumors characterized by amplification or overexpression of CDK4 and/or CCND1.

19-22. (canceled)

23. The method of claim 1, wherein the therapeutically effective amount is from about 100 mg to about 400 mg BID.

24. The method of claim 1, wherein the therapeutically effective amount is about 100 mg.

25. The method of claim 1, wherein the therapeutically effective amount is about 200 mg.
